(19) United States
(12) Patent Application Publication
(10) Pub. No.: US 2012/0295264 A1
(43) Pub. Date: Nov. 22, 2012

(54) METHOD FOR DETECTING ULCERATIVE COLITIS

(75) Inventors: Shigeru Kanaoka, Hamamatsu-shi (JP); Yasushi Hamaya, Hamamatsu-shi (JP); Kenichi Yoshida, Hamamatsu-shi (JP)

(73) Assignees: OLYMPUS CORPORATION, Tokyo (JP); NATIONAL UNIVERSITY CORPORATION HAMAMATSU UNIVERSITY SCHOOL OF MEDICINE, Hamamatsu-shi (JP)

(21) Appl. No.: 13/567,625

(22) Filed: Aug. 6, 2012

Related U.S. Application Data
(63) Continuation of application No. PCT/JP2011/052620, filed on Feb. 8, 2011.

(30) Foreign Application Priority Data
Feb. 8, 2010 (JP) 2010-025024

(51) Int. Cl. C12Q 1/68

(52) U.S. Cl. 435/6.11

(57) ABSTRACT

An object of the present invention is to provide a method of detecting an inflammatory bowel disease, and particularly ulcerative colitis, by using a component contained in fecal matter as an indicator. Namely, the present invention provides a method for detecting ulcerative colitis comprising: (A) a step of extracting RNA contained in fecal matter collected from a subject, (B) a step of measuring the amount of RNA derived from a marker gene in the RNA obtained in step (A), and (C) a step of comparing the amount of the RNA derived from the marker gene measured in step (B) with a preset threshold value, wherein the marker gene is one or more types of genes selected from the group consisting of COX-2 gene, B2M gene, MMP-7 gene, Snail gene, CD45 gene and CEA gene.

### TECHNICAL FIELD

The present invention relates to a method for detecting ulcerative colitis that utilizes a marker gene. More specifically, the present invention relates to a method for detecting the presence or absence of affection of a subject with ulcerative colitis from which fecal matter was collected, and the disease phase thereof, by using the amount of RNA derived from a marker gene contained in the fecal matter as an indicator thereof.

The present application claims priority on the basis of Japanese Patent Application No. 2010-25024, filed in Japan on Feb. 8, 2010, the contents of which are incorporated herein by reference.

### BACKGROUND ART

Inflammatory bowel disease is the generic name of a disease of unknown etiology that presents with symptoms such as abdominal pain, diarrhea, rectal bleeding, fever, anemia and weight loss, and causes chronic inflammation or ulceration of the mucosal membrane of the digestive tract such as the large and small intestines. Accompanying the increasing prevalence of a Western diet, the number of patients suffering from this disease is continuing to increase in Japan as well. One of the characteristics of inflammatory bowel disease is the large number of young persons affected with this disease. In addition, since there are many cases in which the disease results in a decrease in quality of life (QOL), it has been designated as a special chronic disease by the Ministry of Health, Labor and Welfare of Japan.

Inflammatory bowel disease patients are broadly classified into ulcerative colitis and Crohn's disease. Ulcerative colitis is a diffuse, non-specific inflammation of unknown etiology that affects the colon, and mainly invades the mucosal membrane and frequently causes erosion and ulcers. Normally, it presents with bloody diarrhea and various degrees of general symptoms. In general, it is categorized according to the spread of symptoms (pancolitis, left-sided colitis, proctitis or right-sided or segmental colitis), disease phase (such as an active phase or remission phase), severity (mild, moderate, severe) or clinical course (relapse-remission type, chronic sustained type, acute fulminant type or initial attack type). On the other hand, Crohn's disease is a disease in which granulomatous lesions accompanied by ulceration and fibrosis occur discontinuously throughout the digestive tract from the oral cavity to the anus. Although varying according to the site and range of the lesions, symptoms include fever, nutritional disorders and anemia, and systemic complications can also occur such as arthritis, iritis or liver disorders. In general, this disease is categorized according to, for example, the location of the lesions (small intestine type, small intestine-large intestine type, rectum type or gastroduodenal type) or the disease phase (such as an active phase or inactive phase) (see, for example, Non-Patent Document 1).

In cases in which the occurrence of either ulcerative colitis or Crohn's disease is suspected on the basis of clinical symptoms, diagnosis is made on the basis of whether or not characteristic lesions are observed. Consequently, in making a diagnosis and determining the course of treatment of these diseases, endoscopic examination plays an important role since it allows direct observation of affected areas while also enabling histopathological examination. However, in cases of endoscopic examinations performed on critical patients, the examination itself may actually cause exacerbation. In addition, there is increasing reluctance to perform endoscopic examinations on the growing number of pediatric patients due to the highly invasive nature of the procedure and the need to perform the examination under anesthesia. Moreover, in order to observe the colon with an endoscope, preliminary treatment using laxatives and the like is required, and since this requires time, the level of acceptance to undergo an endoscopic examination among out-patients is not very high and it is difficult to perform the procedure easily. Consequently, there is a desire for an examination method that is less invasive and offers high sensitivity and specificity.

In addition, ulcerative colitis and Crohn's disease have unknown causes, there is no fundamental therapy and it is difficult to achieve a complete cure. Consequently, there is repeated relapse and remission, thereby considerably impairing patient QOL. Thus, in these diseases, it is important to prolong the remission phase as long as possible and implement treatment promptly when relapse has occurred. In order to accomplish this as well, there is a strong desire for a non-invasive indicator that is effective for predicting disease activity and relapse.

Indicators that include the disease activity index (DAI) and other general symptoms have been previously used to determine the activity of these diseases. However, indicators that reflect the primary location of lesions in the form of lesions of the digestive tract mucosa have been recognized as being important in terms of high sensitivity and specificity.

On the other hand, in diagnosing colon cancer, in which the affected site is also the colon, a method has been disclosed that uses a component contained in fecal matter as an indicator (see, for example, Patent Documents 1 to 3). Since cells that have detached from cancer tissue are contained in fecal matter, the composition of fecal matter is thought to be able to reflect gastrointestinal lesions. Therefore, in this method, cancer patients and normal subjects are distinguished by using a gene as a biomarker that is lowly expressed in normal tissue but is highly expressed in cancer tissue, and using mRNA of that gene present in fecal matter as an indicator. In this manner, by using fecal matter for the specimen, the examination is not invasive and the burden of the examination on the subject can be dramatically improved.

With respect to inflammatory bowel disease as well, various research has been conducted on whether or not components contained in fecal matter can be used as indicators to predict disease activity and relapse. For example, studies have been conducted as to whether or not the amounts of proteins derived from eosinophils such as lactoferrin or calprotectin present in fecal matter reflect mucosal lesions and disease activity, and the amounts of these proteins have been reported to be useful as indicators (see, for example, Non-Patent Documents 2 and 3). However, in the case of using the amounts of these proteins as indicators, they can only be used as a rough assessment for classifying as an active phase or remission phase.

### DESCRIPTION OF THE RELATED ART

### Patent Documents

Patent Document 1: Japanese Patent No. 4134047
Patent Document 2: Japanese Patent No. 4206425
Patent Document 3: International Publication No. WO 2007/018257

### Non-Patent Documents

Non-Patent Document 1: Miyamoto, A., author, Matsukawa M., editor, “Clinical Classification of Gastrointestinal Diseases—Classification at a Glance and Endoscopy Atlas”, Yodosha Co., Ltd., 2008, pp. 136-152.
Non-Patent Document 2: Thomas R. Walker and six others, Journal of Pediatric Gastroenterology and Nutrition, 2007, Vol. 44, pp. 414-422.
Non-Patent Document 3: Ulrika Lorentzon Fagerberg and four others, Journal of Pediatric Gastroenterology and Nutrition, 2007, Vol. 45, pp. 414-420.

### DISCLOSURE OF THE INVENTION

### Problems to be Solved by the Invention

An object of the present invention is to provide a method for detecting inflammatory bowel disease, and in particular ulcerative colitis, by using a component contained in fecal matter as an indicator.

### Means for Solving the Problems

As a result of conducting extensive studies to solve the aforementioned problems, when the inventors of the present invention extracted RNA from fecal matter supplied from patients with ulcerative colitis and analyzed RNA derived from a human gene contained in that RNA, the inventors of the present invention found that the presence of affection with ulcerative colitis and the disease phase thereof, whether it be the active phase, inactive phase or remission phase, can be discriminated comparatively precisely by using the amount of RNA derived from a specific gene contained in fecal matter as an indicator, thereby leading to completion of the present invention.

Namely, the present invention employs the composition described below.

(1) A method for detecting ulcerative colitis by using a marker gene of ulcerative colitis, comprising:

(A) a step of extracting RNA contained in fecal matter collected from a subject,

(B) a step of measuring the amount of RNA derived from a marker gene in the RNA obtained in step (A), and

(C) a step of comparing the amount of the RNA derived from the marker gene measured in step (B) with a preset threshold value; wherein,

the marker gene is one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene.

(2) The method for detecting ulcerative colitis described in (1) above, wherein the threshold value is a threshold value for distinguishing between an active phase ulcerative colitis affected group and a normal subject group, and

the step (C) is a step of assessing the magnitude of the possibility that the subject is affected with active phase ulcerative colitis.

(3) The method for detecting ulcerative colitis described in (1) above, wherein the step (C) is:

(C′1) a step of comparing the amount of the RNA derived from a marker gene measured in step (B) with a preset first threshold value and/or a second threshold value, and assessing the magnitude of the possibility that the disease phase of the subject is any of an active phase, inactive phase or remission phase of ulcerative colitis:

wherein, the first threshold value is a threshold value for distinguishing between an inactive phase or remission phase affected group and an active phase affected group, and

the second threshold value is a threshold value for distinguishing between an active phase and inactive phase affected group and a remission phase affected group.

(4) The method for detecting ulcerative colitis described in (1) above, wherein the subject is a person affected with remission phase or inactive phase ulcerative colitis, the step (C) is:

(C′2) a step of comparing the amount of the RNA derived from a marker gene measured in step (B) with a preset second threshold value, and predicting that the ulcerative colitis of the subject has relapsed in the case the amount of the RNA derived from the marker gene exceeds the second threshold value;

wherein, the second threshold value is a threshold value for distinguishing between an active phase or inactive phase affected group and a remission phase affected group.

(5) A method for detecting ulcerative colitis by discriminating between active phase ulcerative colitis and colon cancer, comprising:

(a) a step of extracting RNA contained in fecal matter collected from a subject,

(b) a step of measuring the amount of RNA derived from cyclooxygenase-2 (COX-2) gene and the amount of RNA derived from carcinoembryonic antigen (CEA) gene present in the RNA obtained in step (a), and

(c) a step of comparing a value obtained by dividing the amount of RNA derived from COX-2 gene by the amount of RNA derived from CEA gene measured in step (b) with a preset threshold value, and assessing the possibility that the subject is in an active phase of ulcerative colitis.

(6) A gene marker of ulcerative colitis that is one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene.
(7) A method for monitoring a disease phase of ulcerative colitis using a marker gene of ulcerative colitis, comprising:

sampling fecal matter from a subject over time, and carrying out the following steps on each of the collected fecal matter:

(A′) a step of extracting RNA contained in the fecal matter,

(B) a step of measuring the amount of RNA derived from a marker gene present in the RNA obtained in step (A′), and

(C″1) a step of comparing the amount of RNA derived from the marker gene measured in step (B) with a preset first threshold value and/or a second threshold value, and assessing the magnitude of the possibility that the disease phase of the subject is any of an active phase, inactive phase or remission phase of ulcerative colitis at each time fecal matter was collected; wherein,

the marker gene is one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene,

the first threshold value is a threshold value for distinguishing between an inactive phase or remission phase affected group and an active phase affected group, and

the second threshold value is a threshold value for distinguishing between an active phase or inactive phase affected group and a remission phase affected group.

(8) A method for screening candidate compounds having anti-ulcerative colitis activity by using a marker gene of ulcerative colitis, comprising:

(P) a step of extracting RNA contained in fecal matter collected from an animal administered a candidate compound,

(Q) a step of measuring the amount of RNA derived from a marker gene present in the RNA obtained in step (P), and

(R) a step of comparing the amount of the RNA derived from the marker gene measured in step (Q) with a preset threshold value; wherein,

the marker gene is one or more types of genes selected from the group consisting of cyclooxygenase-2 (COX-2) gene, 132 microglobulin (B2M) gene, matrix metalloproteinase-7 (MMP-7) gene, Snail gene, CD45 gene and carcinoembryonic antigen (CEA) gene.

### EFFECTS OF THE INVENTION

Use of the method for detecting ulcerative colitis of the present invention enables highly accurate detection of active phase ulcerative colitis by using fecal matter collected from a subject as a specimen. Consequently, carrying out the method for detecting ulcerative colitis of the present invention on fecal matter collected from a specimen makes it possible to detect whether or not the subject is affected with ulcerative colitis more safely and accurately. In addition, in the case the subject has been preliminarily diagnosed to be affected with ulcerative colitis, the disease phase of the subject, and particularly whether or not the subject is in the active phase, can be determined.

### BRIEF DESCRIPTION OF THE DRAWINGS

FIG. 1 is a drawing showing the number of copies of COX-2 mRNA contained per 0.025 μg of RNA extracted from a fecal sample collected in Example 1 for each disease phase (activity) of ulcerative colitis.

FIG. 2 is a drawing showing the number of copies of B2M mRNA contained per 0.025 μg of RNA extracted from a fecal sample collected in Example 1 for each disease phase (activity) of ulcerative colitis.

FIG. 3 is a drawing showing the number of copies of MMP-7 mRNA contained per 0.025 μg of RNA extracted from a fecal sample collected in Example 1 for each disease phase (activity) of ulcerative colitis.

FIG. 4 is a drawing showing the number of copies of Snail mRNA contained per 0.025 μg of RNA extracted from a fecal sample collected in Example 1 for each disease phase (activity) of ulcerative colitis.

FIG. 5 is a drawing showing the number of copies of CD45 mRNA contained per 0.025 μg of RNA extracted from a fecal sample collected in Example 1 for each disease phase (activity) of ulcerative colitis.

FIG. 6 is a drawing showing the number of copies of CEA mRNA contained per 0.025 μg of RNA extracted from a fecal sample collected in Example 1 for each disease phase (activity) of ulcerative colitis.

FIG. 7 is a drawing showing the value of COX-2/CEA (value obtained by dividing the number of copies of COX-2 mRNA by the number of copies of CEA mRNA) contained per 0.025 μg of RNA extracted from a fecal sample collected in Example 2 for each disease.

FIG. 8 is a drawing indicating analyzing receiver operating characteristic (ROC) values in the case of using the ratio of the amount of RNA derived from each marker gene obtained in Examples 2 and 3 as a discrimination marker for active phase ulcerative colitis and colon cancer.

FIG. 9 is a drawing showing the number of bowel movements per day of ulcerative colitis patients, drug administration status and the amount (the number of copies) of RNA derived from each marker gene in Example 4.

### BEST MODE FOR CARRYING OUT THE INVENTION

In the present invention and present description, the active phase, inactive phase and remission phase of ulcerative colitis respectively refer to the states indicated below.

Active phase: Complaints of bloody feces, absence of mucosal vascular patterns endoscopically, and hemorrhaging, erosion or ulceration.

Inactive phase: Absence of bloody feces, active phase findings not completely absent endoscopically (although mucosal vascular patterns have appeared, some mild erythema is observed).

Remission phase: Absence of bloody feces, absence of active phase findings endoscopically, and appearance of mucosal vascular patterns.

In addition, in the present invention and present description, “RNA derived from a marker gene” refers to RNA transcribed from all or a portion of genome DNA of a marker gene, and may be mRNA of that gene or a portion (fragment) of that mRNA.

In the present invention and present description, a “non-affected person” refers to a person not affected with ulcerative colitis, and includes not only normal subjects, but also persons affected with a disease other than ulcerative colitis.

The present invention is characterized by the use of one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene as genetic markers of ulcerative colitis. These six types of genes are hereinafter referred to as marker genes of ulcerative colitis. One type of these genes may be used as a marker gene or two or more types may be used in combination. In the case of using two or more types of these genes in combination, ulcerative colitis can be detected more accurately in examinations.

These marker genes demonstrate a strong positive correlation between the amounts of RNA derived from those genes and the activity of ulcerative colitis. Namely, RNA derived from these marker genes is contained in significantly greater amounts in fecal matter of persons affected with active phase ulcerative colitis than in fecal matter of non-affected persons (such as normal subjects). In addition, the amounts of RNA derived from these marker genes in fecal matter tend to increase in the order of remission phase, inactive phase and active phase in an ulcerative colitis affected group. Moreover, the amounts of RNA derived from these marker genes in fecal matter tends to increase as the area of the affected site in the colon becomes larger.

Although the reason why these genes can be used as marker genes is not clear, it is presumed to be as follows. Namely, in persons affected with inflammatory bowel diseases such as ulcerative colitis, a larger number of cells are predicted to detach from the intestinal wall than in non-affected persons, and the number of detached cells is predicted to decrease in a stepwise manner from the active phase to the remission phase. Although RNA derived from various types of genes is contained in cells of the intestinal wall, COX-2 gene, B2M gene, MMP-7 gene, Snail gene, CD45 gene and CEA gene demonstrate a higher correlation between the amount of RNA derived from these genes present in fecal matter and the number of detached cells than the numerous other genes contained in the detached cells, and as a result thereof, the amount of RNA derived from these genes present in fecal matter is presumed to be able to serve as a biomarker of ulcerative colitis.

In the present invention, the amount of RNA derived from a marker gene of ulcerative colitis present in fecal matter is measured, and the presence or absence of affection with ulcerative colitis and the disease phase thereof is examined by using the resulting measured value as an indicator. For example, a threshold value is preliminarily set for the amount of RNA derived from a marker gene present in fecal matter, and whether or not a subject is affected with ulcerative colitis can be assessed from the amount of the RNA derived from the marker gene present in fecal matter collected from a subject based on that threshold value.

The method for detecting ulcerative colitis of the present invention is a method for detecting ulcerative colitis by using a marker gene of ulcerative colitis that comprises the following steps (A) to (C), wherein the marker gene is one or more types of genes selected from the group consisting of COX-2 gene, B2M gene, MMP-7 gene, Snail gene, CD45 gene and CEA gene:

(A) a step of extracting RNA contained in fecal matter collected from a subject,

(B) a step of measuring the amount of RNA derived from a marker gene in the RNA obtained in step (A), and

(C) a step of comparing the amount of the RNA derived from the marker gene measured in step (B) with a preset threshold value.

The following provides an explanation of each step.

First, in step (A), RNA contained in fecal matter collected from a subject is extracted. In this step, the extracted RNA may be purified in accordance with ordinary methods. There are no particular limitations on the methods used to extract and purify RNA from fecal matter, and a commercially available purification kit and the like can be used. Furthermore, prior to proceeding to the next step, the amount and concentration of the RNA obtained in step (A) may be measured in advance. There are no particular limitations on the methods used to measure the amount and concentration of RNA, and spectrophotometry or any other known method in the relevant field may be used.

There are no particular limitations on the fecal matter supplied for extraction of RNA in step (A) provided it is of human origin, and for example, a specimen collected for a periodic medical examination or other health examination and the like can be used. In addition, the fecal matter may be that obtained immediately after voiding or that which has been stored for a fixed period of time after collection. There are no particular limitations on the method used to store the fecal matter, and any storage method applicable to fecal matter, such as that used in clinical laboratory testing, may be used. For example, fecal matter that has been frozen or refrigerated may be used for RNA extraction, or fecal matter that has been stored by immersing or suspending in various types of storage solutions may be used. The storage solution added to the fecal matter is preferably a solution capable of storing fecal matter by inhibiting damage to RNA present in the fecal matter, such as a fecal sample preparation solution having a water-soluble alcohol and the like as an active ingredient thereof (see, for example, PCT Patent Application, International Publication No WO 2010/024251).

The RNA extracted in step (A) may be used directly in step (B) or may be used in step (B) after storing for a fixed period of time. Any method may be used to store the RNA provided it is a method that enables storage while inhibiting decomposition of the RNA, and for example, the RNA may be stored after freeze-drying or may be stored in the state of a solution obtained by dissolving in purified water.

Next, in step (B), the amount of RNA derived from a marker gene present in the RNA obtained in step (A) is measured. There are no particular limitations on the method use to measure the amount of RNA derived from the marker gene in step (B), and can be suitably selected from known techniques commonly used in the case of measuring amounts of nucleic acids having a specific nucleotide sequence.

Furthermore, in the present invention and description, measurement of the amount of RNA does not refer to a precise measurement, but rather may refer to semi-quantitative measurement to a degree that enables a qualitative comparison with a prescribed threshold value and the like. For example, RNA derived from a gene marker can be detected by a known technique employed in the relevant technical field, and the amount of RNA can be calculated from the resulting detection results based on a calibration curve prepared from the results of detecting a control sample having a known concentration. There are no limitations on the method used to detect the RNA derived from a marker gene, and any method may be used that is known in the relevant technical field. For example, RNA may be detected by a hybridization method using a probe capable of hybridizing with RNA derived from a marker gene, or RNA may be detected by a method that uses a primer capable of hybridizing with RNA derived from a marker gene and a polymerase. Commercially available detection kits and the like can also be used.

In the case of the measurement of step (B), RNA derived from a marker gene present in the RNA obtained in step (A) may be directly detected quantitatively, or RNA derived from a marker gene in that RNA may be detected quantitatively after having amplified by a nucleic acid amplification reaction. For example, RNA derived from a marker gene can be detected directly by a northern blotting method consisting of using two probes that hybridize adjacent to the RNA derived from a gene marker, binding the probes by a ligase reaction following hybridization, and quantitatively detecting the resulting conjugate, or a method consisting of carrying out northern blotting using a labeled probe, and detecting the amount of probe that has formed an aggregate by hybridization using the label as an indicator.

Since the amount of RNA derived from a marker gene is extremely small, it can also be measured by a method that uses a nucleic acid amplification method. For example, after having synthesized cDNA by carrying out a reverse transcription reaction on all or a portion of the RNA obtained in step (A), RNA derived from a marker gene can be detected and the amount thereof can be measured by carrying out nucleic acid amplification using the resulting cDNA as a template. Although a polymerase chain reaction (PCR) is normally carried out to amplify nucleic acid by using cDNA as a template, loop-mediated isothermal amplification (LAMP) or isothermal and chimeric primer-initiated amplification of nucleic acids (ICAN) can also be used. In addition, detection of RNA derived from a marker gene and quantification thereof can be easily carried out simultaneously by carrying out semi-quantitative PCR such as real-time PCR for the nucleic acid amplification method. Alternatively, RNA derived from a marker gene can also be amplified by nucleic acid sequence-based amplification (NASBA) in which RNA is amplified directly from RNA. The amplification product of the RNA derived from a marker gene can be quantified by a method known in the relevant technical field. For example, the amplification product can be measured quantitatively by suitably and specifically isolating the amplification product by gel or capillary electrophoresis followed by detecting the amplification product.

In addition, various types of sensitization methods such as Invader™ can also be used to detect RNA derived from a marker gene. A sensitization method can be used both in the case of directly detecting RNA derived from a marker gene present in the RNA obtained in step (A) and in the case of detecting the RNA after having amplified by a nucleic acid amplification reaction.

In the case of using two or more types of marker genes in combination, the amounts of each RNA derived from the marker genes may be measured separately, or the amounts thereof may be measured simultaneously. For example, amplification products may be obtained by carrying out PCR for each type of gene by using for the template cDNA obtained by a reverse transcription reaction from all of a portion of the RNA obtained in step (A), or a plurality of gene amplification products may be obtained in a single reaction by carrying out multiplex PCR.

Following step (B), the amount of the RNA derived from a marker gene measured in step (B) is compared with a preset threshold value in step (C). The presence or absence of the possibility that the subject is affected with ulcerative colitis and the magnitude of that possibility can be assessed as a result of that comparison. More specifically, in the case, for example, the amount of RNA derived from a gene marker measured in step (B) is greater than a preset threshold value, the subject can be assessed to be affected (or have a high possibility of being affected) with ulcerative colitis, while in the case the amount of RNA is below the threshold value, the subject can be assessed to not be affected (or have a low possibility of being affected) with ulcerative colitis.

The threshold value used at this time can be suitably set by a person with ordinary skill in the art by carrying out required preliminary examinations and the like in consideration of, for example, the type of method used to measure the RNA derived from a gene marker in step (B). For example, by determining the amounts of RNA derived from a gene marker using the same measurement method as step (B) for fecal matter collected from a group known to not be affected with ulcerative colitis (non-affected group) and fecal matter collected from a group known to be affected with ulcerative colitis (affected group), and comparing the measured values of both groups, a threshold value can be suitably set for discriminating between both groups.

Since the amount of RNA derived from COX-2 gene contained in fecal matter demonstrates a strong correlation with the activity of ulcerative colitis, in the method for detecting ulcerative colitis of the present invention, it is extremely effective for detecting persons affected with active phase ulcerative colitis. More specifically, after setting a threshold value for distinguishing between an active phase ulcerative colitis affected group and a normal group, whether or not a subjected is affected with active phase ulcerative colitis is assessed using that threshold value in step (C). In the case the amount of RNA derived from a marker gene measured in step (B) is greater than the threshold value, the subject can be assessed to be affected (or have a high possibility of being affected) with active phase ulcerative colitis.

In this manner, according to the method for detecting ulcerative colitis of the present invention, since an assessment can be made as to whether or not a subject is affected with ulcerative colitis, or as to the magnitude of the possibility of that subject being affected with ulcerative colitis, the method for detecting ulcerative colitis of the present invention can be preferably used for primary screening for ulcerative colitis during periodic medical examinations and the like.

Desired detection accuracy can also be taken into consideration when setting the threshold value. In the case the distribution of amounts of RNA derived from a gene marker present in fecal matter has been clearly determined for both a non-affected group and an affected group, a threshold value can be set so that the probability of the amount of RNA derived from a marker gene present in fecal matter collected from a person affected with ulcerative colitis is below the threshold value (namely, the probability of being a non-affected person) is within a desired range (such as 10% or less, preferably 5% or less, more preferably 2.5% or less, even more preferably 1% or less, and particularly preferably 0%).

In addition, in the case the distribution of the amounts of RNA derived from a gene marker present in fecal matter has been clearly determined for only a non-affected group, such as the case in which a subject is a non-affected person, the threshold value can be set so that the amount of RNA derived from a gene marker present in fecal matter collected from the subject is a desired value in terms of a percentile of non-affected persons (for example, 90 percentile, preferably 95% percentile, more preferably 97.5 percentile, even more preferably 99 percentile, and particularly preferably 100 percentile). In addition, the threshold value can be set so that a significance probability (P value) that the amount of RNA derived from a gene marker present in fecal matter collected from the subject is below the threshold value is a desired value (for example, 10%, preferably 5%, more preferably 1% and even more preferably 0.1%). Furthermore, the P value may be a two-sided probability or one-side probability. The threshold value can be set in the same manner in the case the distribution of the amounts of RNA derived from a marker gene present in fecal matter has only been clearly determined for an affected group. Furthermore, the P value can be determined according to a statistical technique such as the Mann-Whitney U test.

Sensitivity and specificity in the method for detecting ulcerative colitis of the present invention can be suitably adjusted with the set threshold value. For example, in the case of desiring to obtain sufficiently high sensitivity, namely in the case of attempting to detecting the presence of ulcerative colitis, the threshold value is preferably set so that the probability that the amount of RNA derived from a marker gene present in fecal matter collected from a person affected with ulcerative colitis is below the threshold value (namely, the probability of being assessed as a non-affected person) is 1% or less and particularly preferably 0%. On the other hand, in the case of using for primary screening in a periodic medical examination and the like, high specificity is preferable even if sensitivity is sacrificed to some extent. Consequently, the threshold value can be set so that, for example, the probability of the amount of RNA derived from a marker gene present in fecal matter collected from a normal subject exceeds the threshold value (namely, the probability of being assessed as an affected person) is, for example, 10% or less and preferably 5% or less. In this manner, in the method for detecting ulcerative colitis of the present invention, the threshold value can be set according to the desired levels of sensitivity and specificity.

As was previously described, the amount of RNA derived from COX-2 gene contained in fecal matter can be used as an indicator of the disease phase of ulcerative colitis. More specifically, in the case a subject is affected with ulcerative colitis, the disease phase thereof can be examined by carrying out the following step (C′1) instead of step (C):

(C′1) a step of comparing the amount of the RNA derived from a marker gene measured in step (B) with a preset first threshold value and/or a second threshold value, and assessing the magnitude of the possibility that the disease phase of the subject is any of an active phase, inactive phase or remission phase of ulcerative colitis.

The first threshold value used in step (C′1) is a threshold value for distinguishing between an inactive phase or remission phase, and an active phase. In other words, this threshold value is for discriminating between persons affected with active phase ulcerative colitis and those affected with other phases of ulcerative colitis. Accordingly, in the case the amount of RNA derived from a marker gene measured in step (B) is greater than the first threshold value, the subject can be assessed to be a person affected with active phase ulcerative colitis (or have a high possibility of being a person affected with active phase ulcerative colitis).

On the other hand, the second threshold value is a threshold value for distinguishing between an active phase or inactive phase, and a remission phase. In other words, this threshold value is for discriminating between persons affected with remission phase ulcerative colitis and persons affected with other phases of ulcerative colitis. However, there are many cases in which it is difficult to clearly differentiate persons affected with remission phase ulcerative colitis and non-affected persons depending on the type of marker gene or type of measurement method used in step (B). Accordingly, in the case the amount of RNA derived from a marker gene measured in step (B) is smaller than the second threshold value, the subject can be assessed to be in the remission phase provided the subject is a person affected with ulcerative colitis or the subject can be assessed to be a non-affected person (or have a high possibility thereof).

Furthermore, in step (C′1), assessment may be made using only the first threshold value or using only the second threshold value, or both the first and second threshold values may be used to make the assessment.

COX-2 gene, B2M gene, MMP-7 gene, Snail gene, CD45 gene and CEA gene all demonstrate statistically significant different amounts of RNA derived from these genes in fecal matter between persons affected with active phase ulcerative colitis and persons affected with remission phase ulcerative colitis or non-affected persons. However, there are also cases in which statistically significant differences are not observed in the amounts of RNA derived from these genes in fecal matter between persons affected with active phase ulcerative colitis and persons affected with remission phase ulcerative colitis or non-affected persons depending on the type of marker gene and the type of measurement method used in step (B).

The first or second threshold value can be set in the same manner as the threshold value used in step (C). More specifically, by determining the amounts of RNA derived from a marker gene according to the same measurement method as step (B) for fecal matter collected from an active phase affected group and fecal matter collected from an other phase affected group, both of which consist of persons with ulcerative colitis for which the disease phase is known, and then comparing the measured values of both groups, a threshold value can be suitably set for discriminating between the two groups. Similarly, by determining the amounts of RNA derived from a marker gene according to the same method as step (B) for fecal matter collected from a remission phase affected group and another phase affected group, both of which consist of persons affected with ulcerative colitis for which the disease phase is known, and then comparing the measured values of both groups, a threshold value can be suitably set for discriminating between the two groups.

In this manner, since the disease phase of persons affected with ulcerative colitis can be identified, the method for detecting ulcerative colitis of the present invention can be of help in, for example, diagnosing the effects of drug therapy or other therapy or determining the administration period. For example, when the method for detecting ulcerative colitis of the present invention is carried out over time on a person affected with ulcerative colitis undergoing drug therapy, in the case that person is moving towards remission as a result of that therapy, the amount of RNA derived from a marker gene contained in fecal matter of that person affected with ulcerative colitis demonstrates a decreasing trend as the duration of therapy increases.

Consequently, the disease phase of ulcerative colitis can be monitored by employing the method for detecting ulcerative colitis of the present invention. Namely, by collecting fecal matter from a subject over time, extracting RNA contained in the fecal matter, measuring the amounts of RNA derived from a gene marker present in the resulting RNA, and comparing the measured amounts of RNA derived from a marker gene with the preset first threshold value and/or second threshold value, the magnitude of the possibility of the disease phase of the subject being any of an active phase, inactive phase or remission phase of ulcerative colitis at the time the fecal matter was collected can be assessed. For example, although therapeutic efficacy for a person affected with ulcerative colitis can be definitively diagnosed mainly by endoscopic examination, this examination places a large burden on the patient as a result of being highly invasive and having to be performed frequently. On the other hand, although diagnostic accuracy is inferior to that of endoscopic examinations consisting of viewing the affected area directly, the method for detecting ulcerative colitis of the present invention considerably reduces the burden placed on the patient. Therefore, monitoring can be carried out by using the method for detecting ulcerative colitis of the present invention as a preliminary diagnosis, and by then making a definitive diagnosis by endoscopic examination as necessary, changes in disease phase can be monitored at an adequate frequency while reducing the burden on the patient. Furthermore, extraction of RNA from fecal matter and measurement of the amount of RNA derived from a gene marker can be carried out in the same manner as the aforementioned steps (A) and (B), respectively.

In addition, ulcerative colitis typically undergoes repeated remission and relapse following onset. Namely, in persons affected with ulcerative colitis, even through the activity level thereof may temporarily decrease as a result of treatment and the like (remission phase), the level activity readily increases again (active phase). Consequently, in the treatment of ulcerative colitis, it is important to detect relapse and begin appropriate treatment as quickly as possible. If relapse could be predicted non-invasively at the phase prior to the appearance of bloody feces and other symptoms, then it would be possible to quickly make a definitive diagnosis of relapse. In addition, in order to prevent full-scale relapse, additional treatment and medication can be added. Furthermore, in the present invention and description, relapse of ulcerative colitis refers to a re-increase in the activity level of ulcerative colitis that had previously been in the remission phase or inactive phase.

Since the amounts of RNA derived from the previously described six types of marker genes present in fecal matter demonstrate a strong correlation with the level of activity of ulcerative colitis, they can be used as indicators for predicting relapse of ulcerative colitis. More specifically, by carrying out the following step (C′2) instead of step (C) on a subject in the form of a person affected with remission phase or inactive phase ulcerative colitis, whether or not the ulcerative colitis of that subject has relapsed can be predicted. Furthermore, the second threshold value indicated below is a threshold value for distinguishing between an active phase or inactive phase affected group and a remission phase affected group in the same manner step (C′1):

(C′2) a step of comparing the amount of the RNA derived from a marker gene measured in step (B) with a preset second threshold value, and predicting that the ulcerative colitis of the subject has relapsed in the case the amount of the RNA derived from the marker gene exceeds the second threshold value.

For example, in the case of having collected fecal matter over time from a person affected with ulcerative colitis not exhibiting bloody feces or other symptoms (namely, in the remission phase or inactive phase), and having measured the amount of RNA derived from at least one type of the aforementioned six types of marker genes, in the case the measured amount of RNA has exceeded a second threshold value, it is predicted that the ulcerative colitis has become active, that the subject is no longer in the remission phase but has entered the inactive phase, and that the ulcerative colitis will advance to the active phase if left untreated. Conversely, in the case the measured amount of RNA is below the second threshold value, it can be determined that the level of activity of the ulcerative colitis is low and that the subject is in the remission phase (or has a high possibility of being in the remission phase).

The second threshold value used in step (C′2) can be set in the same manner as that used in step (C′1). Alternatively, the second threshold value used in step (C′2) can also be set corresponding to the individual subject. For example, by measuring the amount of RNA derived from a marker gene over time for a single ulcerative colitis patient, the threshold value can be set so as to distinguish between the remission phase and the active phase or inactive phase of the ulcerative colitis of that patient.

In addition, the method for detecting ulcerative colitis of the present invention can also be used to assess drug efficacy when screening for candidate compounds of drugs used to treat ulcerative colitis (anti-ulcerative colitis drugs). For example, by extracting RNA contained in fecal matter collected from an animal administered a candidate compound, measuring the amount of RNA derived from a marker gene in the resulting RNA, and comparing the measured amount of RNA derived from a marker gene with a preset threshold value, the presence or absence of ulcerative colitis in the animal, or the disease phase thereof, can be assessed. For example, in the case of administering a candidate compound to a model animal affected with active phase ulcerative colitis, followed by investigating the disease phase of the ulcerative colitis by collecting fecal matter, in the case the model animals has been assessed as being in the inactive phase or remission phase, the administered candidate compound can be assessed to have anti-ulcerative colitis activity. Furthermore, the animal administered the candidate compound may be an animal used as a study animal, such as a mouse, rat, rabbit, dog or monkey, or a human.

As is described in Patent Documents 1 to 3, the amount of RNA derived from COX-2 gene contained in fecal matter can be used as an indicator of the presence or absence of affection with colon cancer. Accordingly, in the case the amount of RNA derived from COX-2 gene contained in fecal matter is significantly greater than that of a normal subject, the subject from which the fecal matter was collected is judged to have a high possibility of being affected with ulcerative colitis or colon cancer. A definitive diagnosis can then be made for the subject as to whether or not the subject is affected with ulcerative colitis or colon cancer by performing an endoscopic examination.

However, when considering the invasiveness of endoscopic examinations, it is desirable to be able to identify ulcerative colitis and colon cancer by a genetic analysis of fecal matter. Therefore, as a result proceeding with further studies, the inventors of the present invention further found that active phase ulcerative colitis and colon cancer can be identified at a high level of accuracy not found in the prior art based on the ratio of the amounts of RNA derived from different types of marker genes.

More specifically, active phase ulcerative colitis and colon cancer can be identified and ulcerative colitis can be detected using the following steps (a) to (c):

(a) a step of extracting RNA contained in fecal matter collected from a subject,

(b) a step of measuring the amount of RNA derived from COX-2 gene and the amount of RNA derived from CEA gene present in the RNA obtained in step (a), and

(c) a step of comparing a value obtained by dividing the amount of RNA derived from COX-2 gene by the amount of RNA derived from CEA gene measured in step (b) with a preset threshold value.

Steps (a) and (b) can be carried out in the same manner as the aforementioned steps (A) and (B).

In step (c) following step (b), a value obtained by dividing the amount of RNA derived from COX-2 gene by the amount of RNA derived from CEA gene measured in step (b) is compared with a preset threshold value. An assessment can then be made as to whether or not the subject is in the active phase of ulcerative colitis based on the result of this comparison. More specifically, in the case the value obtained by dividing the amount of RNA derived from COX-2 gene by the amount of RNA derived from CEA gene (to be described as the “value of COX-2/CEA”) is greater than a preset threshold value, then the subject can be assessed has not being affected with colon cancer but being the active phase of ulcerative colitis (or having a high possibility thereof), while in the case the aforementioned value is below the threshold value, the subject can be assessed as not being affected with active phase ulcerative colitis (or having a high possibility thereof). Furthermore, the subject is assessed as not being affected with ulcerative colitis in the case RNA derived from CEA gene is not present in fecal matter, or is contained at a level below the detection limit of the measurement and the amount of RNA derived from CEA gene cannot be measured in step (b).

The threshold value used in step (c) can be suitably set by a person with ordinary skill in the art by carrying out required preliminary examinations and the like in consideration of, for example, the type of measurement method used in step (b). For example, by determining the amounts of RNA derived from COX-2 gene and RNA derived from CEA gene using the same measurement method as step (b) for fecal matter collected from a group known to be in the active phase of ulcerative colitis (active phase ulcerative colitis group) and fecal matter collected from a group known to be affected with colon cancer (colon cancer group), followed by comparing the values of COX-2/CEA for both groups, a threshold value can be suitably set for discriminating between both groups.

In addition, when setting the threshold value used in step (c), a desired detection accuracy can be taken into consideration in the same manner as the threshold value used in step (C).

For example, active phase ulcerative colitis and colon cancer can be identified with even higher accuracy by setting the threshold value to within the range of 5 to 100 and preferably 10 to 40.

The amounts of RNA derived from the CD45 gene, B2M gene, MMP-7 gene and Snail gene of the six types of gene markers for ulcerative colitis of the present invention are also known to increase in colon cancer in the same manner as the value of COX-2/CEA (Patent Documents 1 to 3). A ratio of the contained amounts of RNA derived from these genes present in fecal matter can be used as markers for identifying active phase ulcerative colitis and colon cancer in the same manner as the value of COX-2/CEA by selecting a suitable combination of two types of genes from among these genes. More specifically, a subject can be assessed as having a high possibility of being affected with ulcerative colitis or colon cancer by using a gene that is a gene marker of ulcerative colitis and in which RNA derived from that gene is contained in a detectable amount in fecal matter collected from a normal subject as a first marker gene, using a gene that is a gene marker of ulcerative colitis and a gene marker of colon cancer as a second marker gene, and using the ratio of the amount of RNA derived from the second marker gene to the amount of RNA derived from the first marker gene contained in fecal matter collected from the subject ([amount of RNA derived from second marker gene]/[amount of RNA derived from first marker gene]) as an indicator thereof.

When determining the ratio of the contents of RNA in fecal matter, in the case the first marker gene serving as the denominator is such that RNA derived from that gene is not contained in fecal matter collected from a normal subject or is only contained at an extremely low level below the measurement limit (namely, in the case the measured value is 0), since cases in which this content ratio cannot be detected inherently cannot be ignored, such an indicator is unlikely to yield statistically reliable results and is not valid as a clinically useful marker. Consequently, a gene that yields a detectable amount of RNA in fecal matter collected from a normal subject is used for the first marker gene. More specifically, CEA gene or B2M gene is used preferably, while CEA gene is used more preferably for the first marker gene.

On the other hand, a gene that is gene marker of ulcerative colitis and a gene marker of colon cancer is used for the second marker gene. More specifically, CD45 gene, B2M gene (excluding the case B2M gene is used for the first marker gene), MMP-7 gene or Snail gene is preferably used for the second marker gene.

More specifically, a value obtained by dividing the amount of RNA derived from CD45 gene by the amount of RNA derived from CEA gene (to be described as the “value of CD45/CEA”), a value obtained by dividing the amount of RNA derived from B2M gene by the amount of RNA derived from CEA gene (to be described as the “value of B2M/CEA”), a value obtained by dividing the amount of RNA derived from MMP-7 gene by the amount of RNA derived from CEA gene (to be described as the “value of MMP-7/CEA”), a value obtained by dividing the amount of RNA derived from Snail gene by the amount of RNA derived from CEA gene (to be described as the “value of Snail/CEA”), a value obtained by dividing the amount of RNA derived from COX-2 gene by the amount of RNA derived from B2M gene (to be described as the “value of COX-2/B2M”), the value obtained by dividing the amount of RNA derived from CD45 gene by the amount of RNA derived from B2M gene (to be described as the “value of CD45/B2M”), and the value obtained by dividing the amount of RNA derived from Snail gene by the amount of RNA derived from B2M gene (to be described as the “value of Snail/B2M”) can be used as markers for identifying active phase ulcerative colitis and colon cancer. In particular, the values of COX-2/CEA, CD45/CEA, B2M/CEA, MMP-7/CEA, Snail/CEA and CD45/B2M are used preferably, while the values of COX-2/CEA, CD45/CEA, B2M/CEA, Snail/CEA and CD45/B2M are used more preferably.

### EXAMPLES

Although the following provides a more detailed explanation of the present invention by indicating examples thereof, the present invention is not limited to the following examples.

### Example 1

### Fecal Samples

Fecal matter was supplied from 12 active phase, 4 inactive phase and 5 remission phase ulcerative colitis patients. In addition, fecal matter was supplied from 111 colon cancer patients and 140 normal subjects. Informed consent was obtained in advance from these patients and normal subjects either orally or in writing. The ulcerative colitis patients and colon cancer patients were patients that had been definitively diagnosed by endoscopic examination and the like. The specimens (fecal samples) were collected 2 to 4 weeks after endoscopic examination or biopsy and prior to surgery or endoscopic resection. The collected fecal samples were first stored at 4° C. following by transferring to a freezer at −80° C. within 24 hours after the start of storage and storing until the time of RNA extraction treatment.

<Extraction and Purification of RNA from Fecal Samples>

Approximately 0.5 g of frozen fecal sample were added to 3 mL of Isogen (Nippon Gene) in a sterilized 5 mL tube, followed by mixing to homogeneity with a homogenizer. After dispensing approximately 0.7 mL aliquots into sterilized 1.5 mL tubes, the tubes were centrifuged for 5 minutes at 12,000×g and 4° C., and the supernatant was dispensed into new sterilized 1.5 mL tubes. 0.3 mL of Isogen and 0.3 mL of chloroform were respectively added to each of the tubes, and the tubes were then vigorously agitated for 30 seconds with a vortex mixer followed by centrifuging for 15 minutes at 12,000×g and 4° C. The resulting aqueous phase was collected while being careful not to introduce contaminants from the upper surface of the tubes, and then transferred to new 1.5 mL tubes. After adding an equal volume of 70% ethanol solution, the tubes were vigorously agitated for 30 seconds with a vortex mixture. RNA was then extracted and purified from the resulting mixtures (0.7 mL) using the RNeasy Mini Kit (Qiagen). The purified RNA was quantified using the NanoDrop 1000 (NanoDrop Wilmington). The RNA was stored at −80° C. until used in subsequent analyses.

<Measurement of Amounts of RNA Derived from Marker Genes>

cDNA was synthesized in accordance with the manual in reaction solutions having a final volume of 20 μl using 0.125 μg of purified RNA, 250 μg of random hexamer and reverse transcriptase M-MLV (RNaseH⁻; Takara Bio).

By carrying out quantitative real-time PCR using the synthesized cDNA as template, the amounts of cDNA in the cDNA synthesized from RNA derived from each gene present in the fecal matter was quantified for COX-2 gene, B2M gene, MMP-7 gene, Snail gene, CD45 gene and CEA gene. Commercially available products available from Applied Biosystems were respectively used as TaqMan™ primer-probe sets for detecting these marker genes. Furthermore, the probes included in these sets consisted of reporter probes labeled with the fluorescent dye FAM on the 5′ end and with a quencher on the 3′ end. More specifically, sterilized purified water was added to 1 μL, of cDNA solution and 1 μL of 20× TaqMan primers and probe mixture (Applied Biosystems) and adjusted to a final volume of 20 μL for use as PCR reaction solutions. PCR solutions prepared for each of the genes was subjected to nucleic acid amplification (PCR) while measuring fluorescence intensity on a real-time basis using the Model 7500 Fast Real-Time PCR System (Applied Biosystems) under reaction conditions consisting of initially treating for 20 seconds at 95° C. followed by 60 cycles of 3 seconds at 95° C. and 30 seconds at 62° C. Plasmids containing cDNA of each gene were used as controls (standard substances) for calculating the number of copies and were amplified in the same manner.

Statistical processing on the amounts of RNA (the number of copies) derived from the marker genes obtained as a result of measurement was carried out with the Mann-Whitney U test. In addition, all statistical processing was carried out in the form of a two-sided test, and a P value of <0.05 was defined as constituting statistical significance.

Furthermore, since the majority of RNA derived from a marker gene is mRNA derived from that gene, it is hereinafter referred to as mRNA.

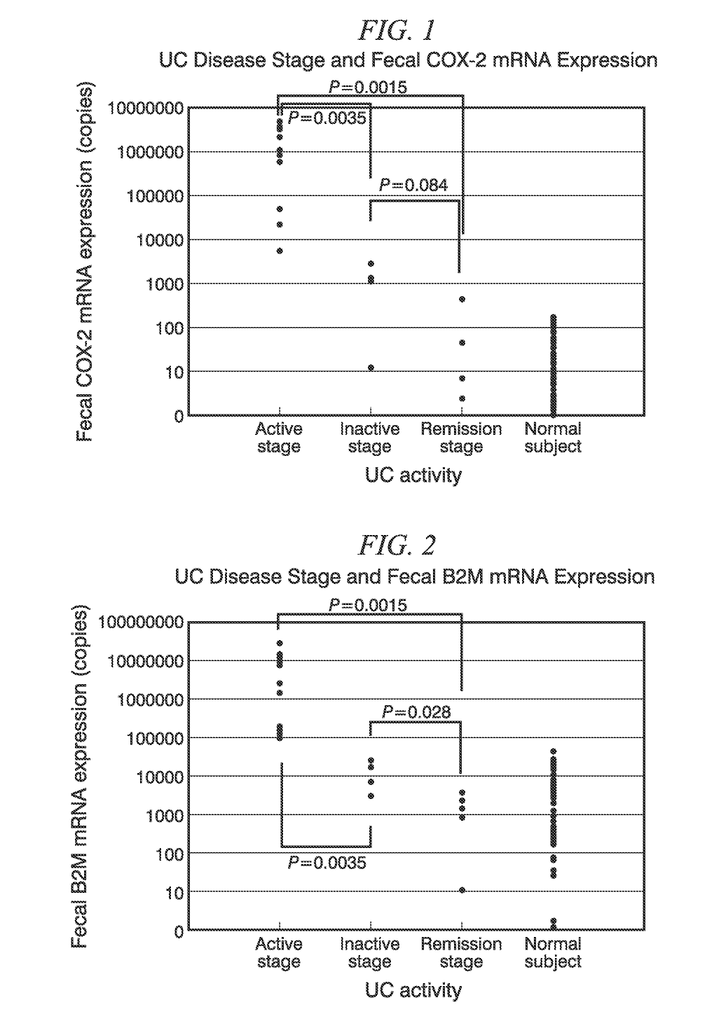

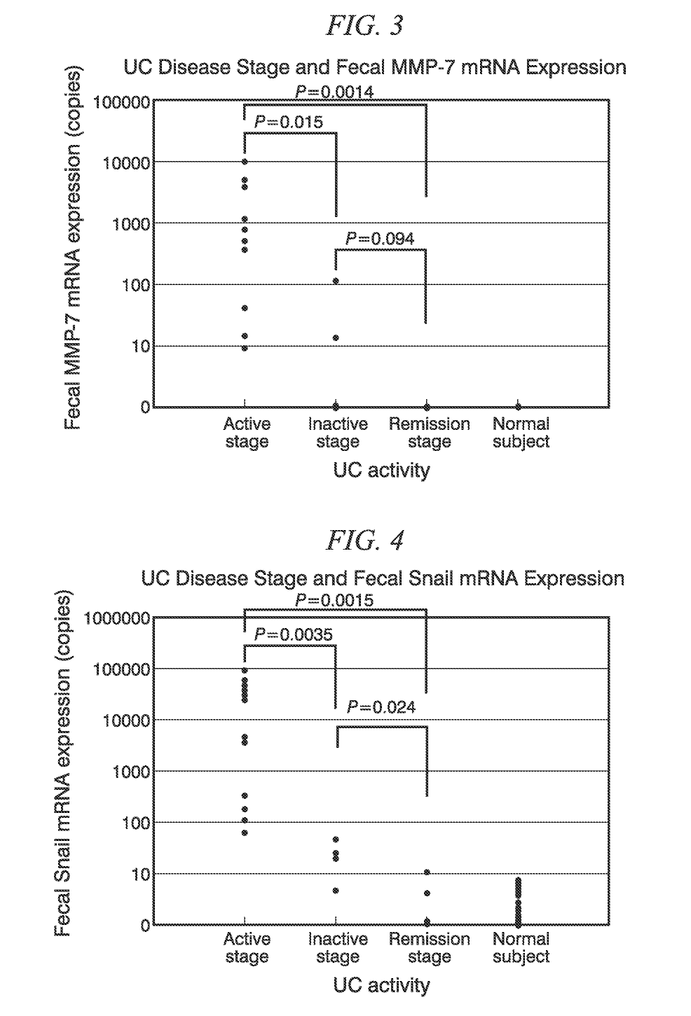

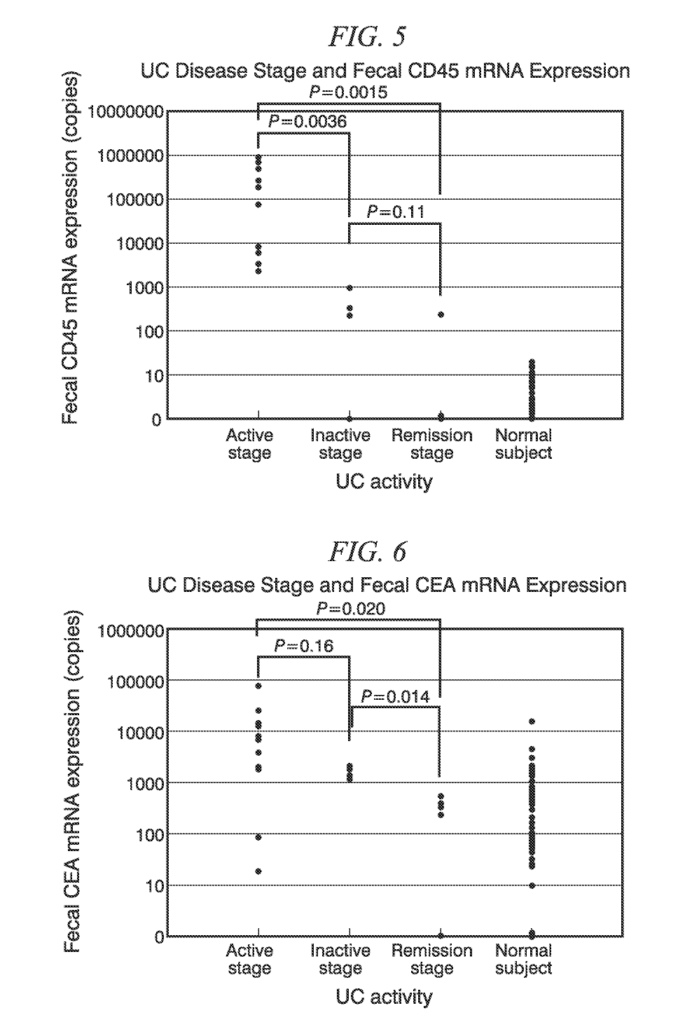

The numbers of copies of mRNA of each marker gene contained in 0.025 μg of RNA extracted from the fecal samples collected from the ulcerative colitis patients and normal subjects are shown in FIGS. 1 to 6 for each disease phase (activity level) of ulcerative colitis. FIG. 1 indicates the results for COX-2 mRNA, FIG. 2 that for B2M mRNA, FIG. 3 that for MMP-7 mRNA, FIG. 4 that for Snail mRNA, FIG. 5 that for CD45 mRNA, and FIG. 6 that for CEA mRNA. In addition, P values between disease phase groups are also shown in the drawings. Moreover, Table 1 respectively indicates the maximum values, minimum values and average values of the numbers of copies of mRNA derived from each of the marker genes for each patient group.

TABLE 1

Amount of mRNA (no. of copies)

| | | |
|---|---|---|
| Minimum | Maximum | Average |

COX-2

| | | | |
|---|---|---|---|
| Active phase group | 4764.5 | 4581340.2 | 1593687.5 |
| Inactive phase group | 12.2 | 2661.3 | 1295.5 |
| Remission phase group | 2.1 | 432.2 | 104.1 |
| Normal subject group | 0.0 | 158.2 | 7.4 |

B2M

| | | | |
|---|---|---|---|
| Active phase group | 68691.1 | 24643106.0 | 6479674.5 |
| Inactive phase group | 2698.3 | 20851.6 | 11128.9 |
| Remission phase group | 9.5 | 3656.3 | 1547.1 |
| Normal subject group | 0.0 | 37670.1 | 3199.0 |

MMP-7

| | | | |
|---|---|---|---|
| Active phase group | 9.7 | 9425.2 | 1841.1 |
| Inactive phase group | 0.0 | 106.9 | 30.1 |
| Remission phase group | 0.0 | 0.0 | 0.0 |
| Normal subject group | 0.0 | 0.0 | 0.0 |

Snail

| | | | |
|---|---|---|---|
| Active phase group | 61.6 | 90534.9 | 24078.9 |
| Inactive phase group | 4.9 | 45.0 | 23.4 |
| Remission phase group | 0.0 | 10.6 | 2.9 |
| Normal subject group | 0.0 | 7.4 | 0.4 |

CD45

| | | | |
|---|---|---|---|
| Active phase group | 2449.4 | 966889.2 | 346035.7 |
| Inactive phase group | 0.0 | 991.8 | 391.8 |
| Remission phase group | 0.0 | 273.5 | 54.7 |
| Normal subject group | 0.0 | 19.0 | 0.9 |

CEA

| | | | |
|---|---|---|---|
| Active phase group | 20.8 | 74791.2 | 12899.4 |
| Inactive phase group | 1270.4 | 2300.1 | 1773.5 |
| Remission phase group | 0.0 | 582.5 | 322.2 |
| Normal subject group | 0.0 | 16215.0 | 589.7 |

As shown in Table 1 and FIGS. 1 to 6, although there is some variation depending on the particular gene, the active phase demonstrated the greatest amount of mRNA, the amounts of mRNA were observed to demonstrate a decreasing trend in the order of the inactive phase, remission phase and normal subjects, and the presence of a correlation was confirmed between the level of activity of ulcerative colitis (UC) and the amount of mRNA for all six genes.

Thus, on the basis of these results, the method for detecting ulcerative colitis of the present invention, which uses one or more types of marker genes selected from the group consisting of COX-2 gene, B2M gene, MMP-7 gene, Snail gene, CD45 gene and CEA gene, is clearly able to detect ulcerative colitis.

### Example 2

The COX-2/CEA value (value obtained by dividing the number of copies of COX-2 mRNA by the number of copies of CEA mRNA) was determined for each fecal sample to investigate the relationship thereof with affection with ulcerative colitis or colon cancer.

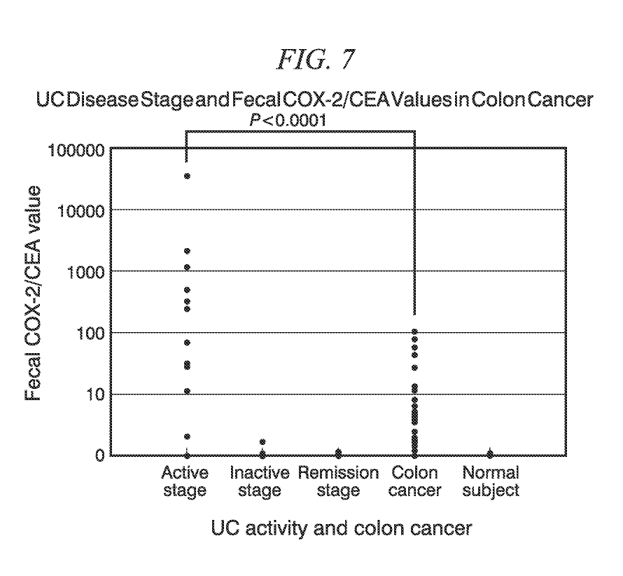

FIG. 7 is a drawing showing the COX-2/CEA values per 0.025 μg of RNA extracted from fecal samples collected from ulcerative colitis patients, colon cancer patients and normal subjects (values obtained by dividing the number of copies of COX-2 mRNA by the number of copies CEA mRNA) for each patient. Furthermore, although the number of copies of CEA mRNA is 0 in the case CEA mRNA was not contained in the fecal samples or was only contained in an amount below the detection limit, in this case, COX-2/CEA values were indicated with a 0. In addition, the COX-2/CEA values of an active phase ulcerative colitis group and colon cancer group are shown in Table 2. Furthermore, due to the large number of samples in the colon cancer group, the numbers of subjects within each range of COX-2/CEA values are also shown. In addition, in the descriptions of the range, the description of “X₁-X₂” refers to the numerical range from a value greater than X₁ to a value of X₂ or less.

TABLE 2

| Active Phase Group | Colon Cancer Group | |
|---|---|---|
| COX-2/CEA Value | Range of COX-2/CEA Values | No. of Subjects |
| 0.7 | 0 | 3 |
| 2.1 | 0-1 | 72 |
| 11.1 | 1-5 | 18 |
| 27.9 | 5-10 | 10 |
| 30.4 | 10-20 | 2 |
| 69.3 | 20-30 | 1 |
| 259.6 | 30-40 | 0 |
| 301.9 | 40-50 | 1 |
| 468.6 | 50-60 | 0 |
| 1198.2 | 60-70 | 2 |
| 2087.4 | 70-80 | 0 |
| 36018.3 | 80-90 | 0 |
| | 90-100 | 2 |

As a result, COX-2/CEA values were nearly 0 in the inactive phase and remission phase ulcerative colitis patient groups in the same manner as the normal subject group. In contrast, in the active phase ulcerative colitis patient group, the minimum value of all 12 samples was 0.7, the maximum value was 36018.3 and the average value was 3373.0. On the other hand, in the colon cancer patient group, the minimum value was 0, the maximum value was 95.4 and the average value was 4.8. On the basis of these results, active phase ulcerative colitis and colon cancer were clearly able to be distinguished from the ratio of the amounts of RNA derived from COX-2 gene and RNA derived from CEA gene present in fecal matter.

In addition, based on the results of Table 2, sensitivity is about 83% (10/12) and specificity is 84% (93/111) in the case of setting the threshold of COX-2/CEA values to 5, sensitivity is about 83% (10/12) and specificity is 93% (103/111) in the case of setting the threshold to 10, and sensitivity is about 75% (9/12) and specificity is 95% (105/111) in the case of setting the threshold to 20, thereby demonstrating that active phase ulcerative colitis can be distinguished from colon cancer and detected in each case. On the other hand, sensitivity is about 58% (7/12) and specificity is about 95% (106/111) in the case of setting the threshold of COX-2/CEA values to 40, while sensitivity is about 50% (6/12) and specificity is about 100% (111/111) in the case of setting the threshold to 100, again demonstrating that active phase ulcerative colitis can be distinguished from colon cancer and detected in each case. On the basis of the above, it is clear that active phase ulcerative colitis can be detected at a high level of accuracy not found in the prior art by setting a suitable threshold value in the method for detecting ulcerative colitis of the present invention.

### Example 3

An investigation was carried out as to whether or not the content ratios of marker genes other than the value of COX-2/CEA can be used as markers for discriminating between active phase ulcerative colitis and colon cancer. The number of copies of CEA gene mRNA or the number of copies of B2M gene mRNA was used for which there are the fewest cases in which the number of copies in the normal subject group is 0 for the denominator when determining content ratio.

Table 3 indicates the values of CD45/CEA of the active phase ulcerative colitis group and colon cancer group, Table 4 indicates the values of B2M/CEA, Table 5 indicates the values of MMP-7/CEA, Table 6 indicates the values of Snail/CEA, Table 7 indicates the values of COX-2/B2M, Table 8 indicates the values of CD45/B2M, Table 9 indicates the values of MMP-7/52M, Table 10 indicates the values of Snail/B2M and Table 11 indicates the values of CEA/B2M. In Tables 3 to 11 as well, due to the large number of samples in the colon cancer group, the numbers of subjects within each range of each of the values are also shown in the same manner as in Table 2. In addition, in the descriptions of the range, the description of “X₁-X₂” refers to the numerical range from a value greater than X₁ to a value of X₂ or less. Moreover, values were indicated with a 0 in the case the denominator in the form of the number of copies of CEA mRNA or the number of copies of B2M mRNA was 0.

TABLE 3

| Active Phase Group | Colon Cancer Group | |
|---|---|---|
| CD45/CEA Value | Range of CD45/CEA Values | No. of Subjects |
| 0.3 | 0 | 13 |
| 1.5 | 0-1 | 91 |
| 3.0 | 1-2 | 2 |
| 4.2 | 2-3 | 2 |
| 7.1 | 3-4 | 3 |
| 22.6 | | |
| 47.7 | | |
| 72.8 | | |
| 107.6 | | |
| 252.9 | | |
| 285.2 | | |
| 9764.0 | | |

TABLE 4

| Active Phase Group | Colon Cancer Group | |
|---|---|---|
| B2M/CEA Value | Range of B2M/CEA Values | No. of Subjects |
| 16.4 | 0 | 1 |
| 30.8 | 0-1 | 2 |
| 45.5 | 1-5 | 44 |
| 59.5 | 5-10 | 28 |
| 117.3 | 10-20 | 22 |
| 598.2 | 20-30 | 6 |
| 1250.9 | 30-80 | 2 |
| 1526.6 | 80-130 | 4 |
| 1677.8 | 130-180 | 1 |
| 3094.0 | 180-230 | 1 |
| 7403.6 | | |
| 112165.2 | | |

TABLE 5

| Active Phase Group | Colon Cancer Group | |
|---|---|---|
| MMP-7/CEA Value | Range of MMP-7/CEA Values | No. of Subjects |
| 0.005 | 0 | 49 |
| 0.005 | 0-0.01 | 20 |
| 0.027 | 0.01-0.1 | 32 |
| 0.038 | 0.1-1.0 | 9 |
| 0.075 | 1.0-2.0 | 1 |
| 0.124 | | |
| 0.126 | | |
| 0.244 | | |
| 0.301 | | |
| 0.611 | | |
| 0.683 | | |
| 45.943 | | |

TABLE 6

| Active Phase Group | Colon Cancer Group | |
|---|---|---|
| Snail/CEA Value | Range of Snail/CEA Values | No. of Subjects |
| 0.01 | 0 | 32 |
| 0.03 | 0-0.01 | 41 |
| 0.08 | 0.01-0.1 | 28 |
| 0.13 | 0.1-0.5 | 10 |
| 0.71 | | |
| 1.77 | | |
| 2.40 | | |
| 2.49 | | |
| 3.68 | | |
| 13.12 | | |
| 16.15 | | |
| 1079.08 | | |

TABLE 7

| Active Phase Group | Colon Cancer Group | |
|---|---|---|
| COX-2/B2M Value | Range of COX-2/B2M Values | No. of Subjects |
| 0.04 | 0 | 3 |
| 0.04 | 0-0.01 | 26 |
| 0.07 | 0.01-0.1 | 39 |
| 0.19 | 0.1-0.2 | 19 |
| 0.24 | 0.2-0.3 | 3 |
| 0.26 | 0.3-0.4 | 8 |
| 0.28 | 0.4-0.5 | 5 |
| 0.31 | 0.5-0.6 | 2 |
| 0.32 | 0.6-0.7 | 3 |
| 0.39 | 0.7-0.8 | 0 |
| 0.43 | 0.8-0.9 | 1 |
| 0.61 | 0.9-1.0 | 1 |
| | 1.0-1.1 | 1 |

TABLE 8

| Active Phase Group | Colon Cancer Group | |
|---|---|---|
| CD45/B2M Value | Range of CD45/B2M Values | No. of Subjects |
| 0.02 | 0 | 13 |
| 0.03 | 0-0.01 | 69 |
| 0.04 | 0.01-0.02 | 16 |
| 0.04 | 0.02-0.03 | 5 |
| 0.05 | 0.03-0.04 | 5 |
| 0.05 | 0.04-0.05 | 2 |
| 0.06 | 0.05-0.06 | 0 |
| 0.06 | 0.06-0.07 | 0 |
| 0.07 | 0.07-0.08 | 0 |
| 0.08 | 0.08-0.09 | 0 |
| 0.09 | 0.09-0.1 | 0 |
| 0.09 | 0.1-0.11 | 1 |

TABLE 9

| Active Phase Group | Colon Cancer Group | |
|---|---|---|
| MMP-7/B2M Value | Range of MMP-7/B2M Values | No. of Subjects |
| 0.00004 | 0 | 49 |
| 0.00006 | 0-0.0001 | 1 |
| 0.00009 | 0.0001-0.001 | 11 |
| 0.00009 | 0.001-0.01 | 44 |
| 0.00010 | 0.01-0.1 | 6 |
| 0.00020 | | |
| 0.00033 | | |
| 0.00040 | | |
| 0.00041 | | |
| 0.00059 | | |
| 0.00107 | | |
| 0.00401 | | |

TABLE 10

| Active Phase Group | Colon Cancer Group | |
|---|---|---|
| Snail/B2M Value | Range of Snail/B2M Values | No. of Subjects |
| 0.001 | 0 | 32 |
| 0.001 | 0-0.001 | 36 |
| 0.001 | 0.001-0.005 | 29 |
| 0.002 | 0.005-0.01 | 9 |
| 0.002 | 0.01-0.02 | 3 |
| 0.002 | 0.02-0.03 | 2 |
| 0.003 | | |
| 0.003 | | |
| 0.003 | | |
| 0.004 | | |
| 0.006 | | |
| 0.010 | | |

TABLE 11

| Active Phase Group | Colon Cancer Group | |
|---|---|---|
| CEA/B2M Value | Range of CEA/B2M Values | No. of Subjects |
| 0.000 | 0 | 1 |
| 0.000 | 0-0.01 | 4 |
| 0.000 | 0.01-0.05 | 10 |
| 0.001 | 0.05-0.1 | 22 |
| 0.001 | 0.1-0.5 | 63 |
| 0.001 | 0.5-1.0 | 9 |
| 0.002 | 1.0-5.0 | 2 |
| 0.009 | | |
| 0.017 | | |
| 0.022 | | |
| 0.032 | | |
| 0.061 | | |

As a result, the values of COX-2/CEA, CD45/CEA, B2M/CEA, MMP-7/CEA, Snail/CEA, COX-2/B2M and CD45/B2M demonstrated different distributions of values between the active phase ulcerative colitis patient group and the colon cancer patient group, thereby suggesting that these groups can be differentiated by setting a suitable threshold value. On the other hand, the values of MMP-7/B2M and CEA/B2M were small in all cases, and the difference in distributions of values between the two groups was not that large.

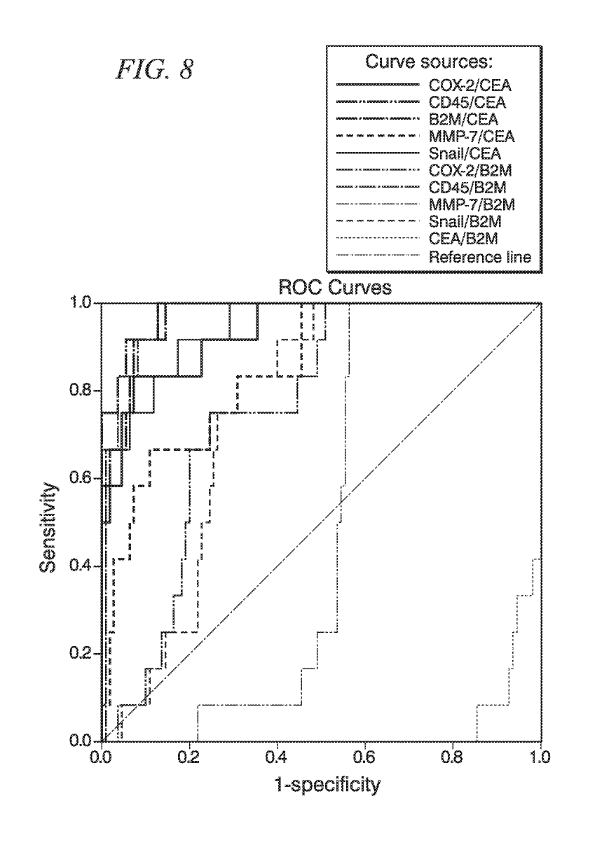

The performance of markers used to differentiate the respective diseases as indicated by the receiver operating characteristic (ROC) was analyzed for the ratios of each of the genes obtained in the present example and the COX-2/CEA values obtained in Example 2. The ROC analysis was prepared using PASW Statistics Ver. 18 (IBM). The number of cases demonstrating a positive effect was 12, the number of cases demonstrating a negative effect was 110 and there was 1 missing value. The results of analysis are shown in Table 12 and FIG. 8. In FIG. 8, ROC curves were obtained by plotting sensitivity on the vertical axis and (1-specificity) on the horizontal axis.

TABLE 12
Area under Curve

| | | | | Asymptotic 95% | |
|---|---|---|---|---|---|
| Test | | | Asymptotic | Confidence Interval | |
| Result | | Standard | Significance | Lower | Upper |
| Variable | Area | Error^{a} | Probability^{b} | Limit | Limit |
| COX-2/CEA | 0.936 | 0.033 | 0.000 | 0.871 | 1.000 |
| CD45/CEA | 0.982 | 0.012 | 0.000 | 0.958 | 1.000 |
| B2M/CEA | 0.970 | 0.016 | 0.000 | 0.940 | 1.000 |
| MMP-7/CEA | 0.850 | 0.052 | 0.000 | 0.749 | 0.951 |
| Snail/CEA | 0.946 | 0.028 | 0.000 | 0.891 | 1.000 |
| COX-2/B2M | 0.758 | 0.053 | 0.003 | 0.654 | 0.863 |
| CD45/B2M | 0.970 | 0.015 | 0.000 | 0.940 | 1.000 |
| MMP-7/B2M | 0.491 | 0.052 | 0.918 | 0.390 | 0.592 |
| Snail/B2M | 0.757 | 0.047 | 0.004 | 0.664 | 0.849 |
| CEA/B2M | 0.030 | 0.016 | 0.000 | 0.000 | 0.060 |

^{a}Based on non-parametric hypothesis
^{b}Null hypothesis: True area = 0.5

As a result, the areas under the curve was 0.5 or more for all of the values of COX-2/CEA, CD45/CEA, B2M/CEA, MMP-7/CEA, Snail/CEA, COX-2/B2M, CD45/B2M and Snail/B2M, and were found to be favorable as markers for detecting and distinguishing active phase ulcerative colitis from colon cancer. In particular, the areas under the curve were 0.8 or more for the values of COX-2/CEA, CD45/CEA, B2M/CEA, MMP-7/CEA, Snail/CEA and CD45/B2M, and were confirmed to be extremely favorable markers.

On the other hand, the areas under the curve were less than 0.5 for the values of MMP-7/B2M and CEA/B2M, and were found to have difficulty in distinguishing between active phase ulcerative colitis and colon cancer at an adequate level of clinical accuracy when used as indicators. In particular, even though the value of B2M/CEA is an extremely favorable marker, the value of CEA/B2M had an extremely low area under the curve of 0.3.

In addition, when the ratio of COX-2 gene to CD45 gene, Snail gene or MMP-7 gene, for which RNA derived from marker gene was unable to be detected in fecal matter in a normal subject group according to the results obtained in Example 1, was investigated as to whether or not it can be used as a marker for distinguishing between active phase ulcerative colitis and colon cancer using the same ROC analysis, the number of cases having a positive effect was 12, the number of cases having a negative effect was 51, and there were 60 missing values. The large number of missing values is due to the large number of cases in which the denominator was 0. In this manner, the number of missing values was excessively large and reliable results were unable to be obtained.

### Example 4

An investigation was carried out on the relationship between the amount of RNA derived from a marker gene of ulcerative colitis of the present in fecal matter and disease phase over time for ulcerative colitis patients having been definitively diagnosed by endoscopic examination and the like.

More specifically, ulcerative colitis patients were observed for the number of bowel movements per day, general condition and the like from Aug. 7, 2002 to Oct. 18, 2002. Moreover, fecal matter was collected from the ulcerative colitis patients on two occasions on August 7 and October 15, and the amounts of RNA (the number of copies) derived from marker genes in the fecal matter were measured for COX-2 gene, B2M gene, MMP-7 gene, Snail gene, CD45 gene and CEA gene in the same manner as Example 1.

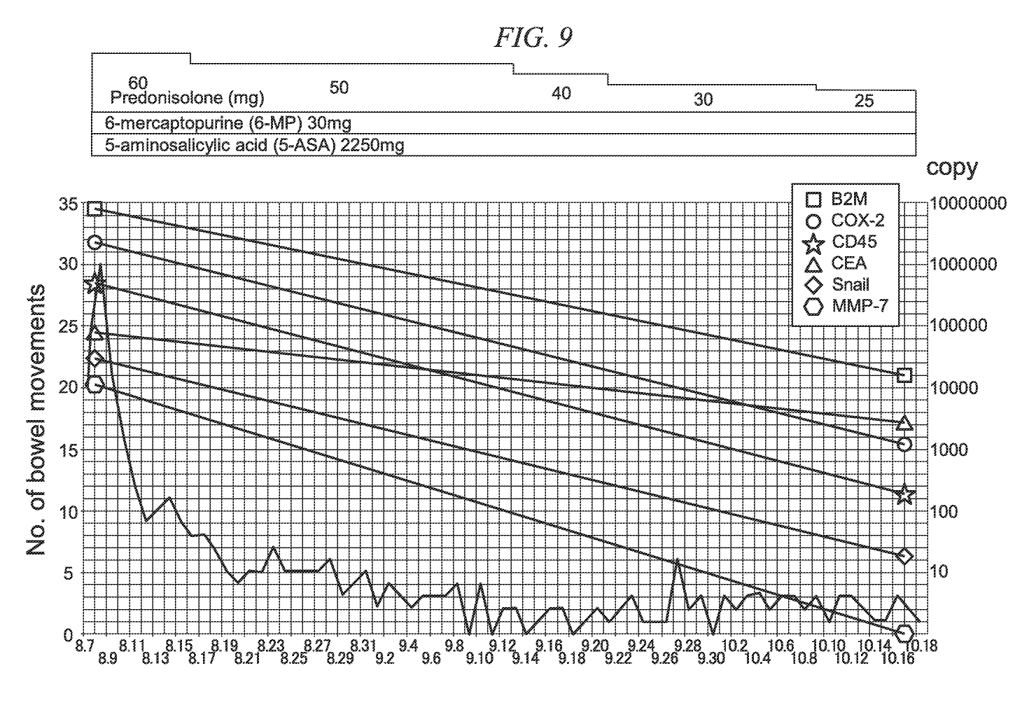

FIG. 9 shows the number of bowel movements per day, drug administration status and the amounts of RNA (the numbers of copies) derived from each marker gene of the ulcerative colitis patients. As shown at the top of FIG. 9, the ulcerative colitis patients underwent drug therapy consisting of prednisolone, 6-mecaptopurine (6-MP) and 5-aminosalicylic acid (5-ASA). As a result of this therapy, the number of bowel movements per day improved from nearly 30 per day at the start of observation to 5 per day or less at completion of the observation period. In addition, the amounts of RNA derived from marker genes in fecal matter demonstrated well-defined decreases for all six types of marker genes from August 7 to October 15. Furthermore, the amounts of RNA derived from the marker genes present in fecal matter are also shown in Table 13.

TABLE 13

| | Aug. 7, 2002 | Oct. 15, 2002 |
|---|---|---|
| COX-2 | 2274712.2 | 1167.8 |
| MMP-7 | 9425.2 | 0.0 |
| Snail | 52793.8 | 21.4 |
| CD45 | 530360.3 | 220.5 |
| B2M | 8770417.0 | 15172.5 |
| CEA | 74791.2 | 2300.1 |

In addition, the disease phases of the ulcerative colitis patients were diagnosed on August 7, August 21 and September 27 by endoscopic examination and observation of general condition. Moreover, general condition was also observed on October 15. Table 14 shows the results of diagnosing the disease phases of ulcerative colitis. A clinical activity index (CAI) score of 6 or higher and a disease activity index (DAI) score of 3 or higher was judged to indicate active phase ulcerative colitis. In addition, a Rachmilewitz endoscopic index (EI) score of 4 or higher was also judged to indicate active phase ulcerative colitis. Disease phase was comprehensively assessed on the basis of these three indices. As a result, as shown in Table 14, since CAI, DAI and EI all indicated an active phase on August 7 and August 21, the ulcerative colitis patients were judged to be in the active phase. In contrast, although DAI and EI scores indicated the active phase on September 27, since CAI was sufficiently low, the ulcerative colitis patients were collectively judged to be in the inactive phase overall. In addition, CAI and DAI scores on October 15 were sufficiently low, and although ulcerative colitis cannot be assessed as being in the remission phase on the basis of these indices alone, since EI scores were not determined, the patients were unable to be assessed as being in the inactive phase or remission phase. However, on the basis of the course of these patients until that time, it was surmised that, as of October 15, there was a high possibility of these ulcerative colitis patients being in the inactive phase rather than the remission phase.

TABLE 14

| | Aug. 7, | Aug. 21, | Sep. 27, | Oct. 15, |
|---|---|---|---|---|
| | 2002 | 2002 | 2002 | 2002 |
| CAI | 12 | 6 | 0 | 0 |
| DAI | 14 | 8 | 5 | 1 |
| EI | 12 | 12 | 7 | — |
| Disease Phase | Active | Active | Inactive | — |

Namely, the ulcerative colitis patients were diagnosed by endoscopic examination and the like as being in the active phase on August 7, and were thought to be in the inactive phase on October 15. Here, although the contents of all six types of marker genes of ulcerative colitis of the present invention in fecal matter tended to increase dependent on the activity of the ulcerative colitis, as shown in FIG. 9, the contents of the marker genes was clearly greater in ulcerative colitis patients who were in the active phase on August 7 than in the patients in the inactive phase (or remission phase) on October 15. Namely, on the basis of these results, since the contents of the marker genes of ulcerative colitis of the present invention in fecal matter fluctuate dependent on the disease phase of the ulcerative colitis, the contents of these marker genes in fecal matter is clearly effective for monitoring the disease phase of ulcerative colitis.

Moreover, ratios of the amounts of RNA derived from each of the marker genes in fecal matter (COX-2/CEA, CD45/CEA, B2M/CEA, Snail/CEA and CD45/B2M values) were calculated. The calculation results are shown in Table 15. As a result, COX-2/CEA values were 30.41 or lower, CD45/CEA values were 7.09 or lower, B2M/CEA values were 117.27 or lower, Snail/CEA values were 0.71 or lower and CD45/B2M values were 0.06 or lower on both August 7 and October 15. When these results are compared with the results of Example 3, the ulcerative colitis patients were assessed as having a high possibility of being affected with ulcerative colitis and not colon cancer, and this assessment result coincides with the results of actual diagnoses by endoscopic examination and the like. Thus, use of the values of COX-2/CEA, CD45/CEA, B2M/CEA, Snail/CEA and CD45/B2M present in fecal matter collected from subjects as indicators clearly makes it possible to assess whether the subjects have a high possibility of being affected with ulcerative colitis or colon cancer.

TABLE 15

| | Aug. 7, 2002 | Oct. 15, 2002 |
|---|---|---|
| COX-2/CEA | 30.41 | 0.51 |
| CD45/CEA | 7.09 | 0.10 |
| B2M/CEA | 117.27 | 6.60 |
| Snail/CEA | 0.71 | 0.01 |
| CD45/B2M | 0.06 | 0.01 |

### INDUSTRIAL APPLICABILITY

Use of the method for detecting ulcerative colitis of the present invention makes it possible to accurately detect the presence or absence of affection with ulcerative colitis and the disease phase thereof. In addition, since the method for detecting ulcerative colitis of the present invention uses fecal matter for the specimen, it is much less invasive than conventional endoscopic examinations, is safe, and reduces the burden of the examination on subjects. Thus, the method for detecting ulcerative colitis of the present invention can be used in clinical laboratory examinations that use fecal samples, and particularly in fields of clinical diagnoses requiring high levels of reliability and safety, including fields such as diagnosis of ulcerative colitis in particular.

1. A method for detecting ulcerative colitis by using a marker gene of ulcerative colitis, comprising:
(A extracting RNA contained in fecal matter collected from a subject,
(B) measuring the amount of RNA derived from a marker gene in the RNA obtained in said (A), and
(C) comparing the amount of the RNA derived from the marker gene measured in said (B) with a preset threshold value; wherein,
the marker gene is one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene.

2. The method for detecting ulcerative colitis according to claim 1, wherein the threshold value is a threshold value for distinguishing between an active phase ulcerative colitis affected group and a normal subject group, and
said (C) is assessing the magnitude of the possibility that the subject is affected with active phase ulcerative colitis.

3. The method for detecting ulcerative colitis according to claim 1, wherein said (C) is:
(C′1) a step of comparing the amount of the RNA derived from a marker gene measured in said (B) with a preset first threshold value and/or a second threshold value, and assessing the magnitude of the possibility that the disease phase of the subject is any of an active phase, inactive phase or remission phase of ulcerative colitis;
wherein, the first threshold value is a threshold value for distinguishing between an inactive phase or remission phase affected group and an active phase affected group, and
the second threshold value is a threshold value for distinguishing between an active phase or inactive phase affected group and a remission phase affected group.

4. The method for detecting ulcerative colitis according to claim 1, wherein the subject is a person affected with remission phase or inactive phase ulcerative colitis, said (C) is:
(C′2) comparing the amount of the RNA derived from a marker gene measured in said (B) with a preset second threshold value, and predicting that the ulcerative colitis of the subject has relapsed in the case the amount of the RNA derived from the marker gene exceeds the second threshold value;
wherein, the second threshold value is a threshold value for distinguishing between an active phase or inactive phase affected group and a remission phase affected group.

5. A method for detecting ulcerative colitis by discriminating between active phase ulcerative colitis and colon cancer, comprising:
(a) extracting RNA contained in fecal matter collected from a subject,
(b) measuring the amount of RNA derived from cyclooxygenase-2 (COX-2) gene and the amount of RNA derived from carcinoembryonic antigen (CEA) gene present in the RNA obtained in said (a), and
(c) comparing a value obtained by dividing the amount of RNA derived from COX-2 gene by the amount of RNA derived from CEA gene measured in said (b) with a preset threshold value, and assessing the magnitude of the possibility that the subject is in an active phase of ulcerative colitis.

6. (canceled)

7. A method for monitoring a disease phase of ulcerative colitis using a marker gene of ulcerative colitis, comprising:
sampling fecal matter from a subject over time, and carrying out the following on each of the collected fecal matter:
(A′) extracting RNA contained in the fecal matter,
(B) measuring the amount of RNA derived from a marker gene present in the RNA obtained in said (A′), and
(C″1) comparing the amount of RNA derived from the marker gene measured in said (B) with a preset first threshold value and/or a second threshold value, and assessing the magnitude of the possibility that the disease phase of the subject is any of an active phase, inactive phase or remission phase of ulcerative colitis at each time fecal matter was collected; wherein,
the marker gene is one or more types of genes selected from the group consisting of cyclooxygenase-2 (COX-2) gene, β2 microglobulin (B2M) gene, matrix metalloproteinase-7 (MMP-7) gene, Snail gene, CD45 gene and carcinoembryonic antigen (CEA) gene,
the first threshold value is a threshold value for distinguishing between an inactive phase or remission phase affected group and an active phase affected group, and
the second threshold value is a threshold value for distinguishing between an active phase or inactive phase affected group and a remission phase affected group.

8. (canceled)

9. (canceled)

10. (canceled)
